(19) United States
(12) Patent Application Publication
(10) Pub. No.: US 2026/0020830 A1
(43) Pub. Date: Jan. 22, 2026

(54) MOTION CONTROL OF A MOVABLE UNIT OF A MEDICAL INSTALLATION

(71) Applicant: Siemens Healthineers AG, Forchheim (DE)

(72) Inventors: Harald KARL, Fuerth (DE); Michael GRIENER, Kemnath (DE); Martin NOLEWAIKA, Erlangen (DE); Martin MEYNDT, Uttenreuth (DE)

(73) Assignee: Siemens Healthineers AG, Forchheim (DE)

(21) Appl. No.: 19/273,678

(22) Filed: Jul. 18, 2025

(30) Foreign Application Priority Data
Jul. 19, 2024 (EP) 24189638.0

(51) Int. Cl. A61B 6/04; A61B 6/00; A61B 6/10

(52) U.S. Cl. CPC A61B 6/0487; A61B 6/102; A61B 6/4441

(57) ABSTRACT

A method for monitoring motion of at least one movable unit of a medical installation, comprises: generating the motion of the unit via at least one actuator; generating motion signals based on an actual motion of the unit and transmitting the motion signals to the control device; checking via the control device; generating error signals via the control device; capturing three-dimensional image data relating to the unit via an image capturing device; generating expectation signals via the control device; checking, via the monitoring device, whether there is a deviation; and generating error signals via the monitoring device.

### CROSS-REFERENCE TO RELATED APPLICATION(S)

The present application claims priority under 35 U.S.C. § 119 to European Patent Application No. 24189638.0, filed Jul. 19, 2024, the entire contents of which are incorporated herein by reference.

### FIELD

One or more example embodiments of the present invention relate to a method for monitoring a motion of at least one movable unit of a medical installation. Moreover, one or more example embodiments of the present invention relate to a corresponding medical installation. Finally, one or more example embodiments of the present invention relate to a control device for such a medical installation.

### BACKGROUND

Medical installations, for example imaging or treatment facilities often involve a mechanical motion of a unit of the installation, for example a patient support table or an imaging unit. In principle, such motions are associated with the risk of collisions that could result in damage to the medical installation or even injury to the patient. In this respect, medical installations are subject to stringent requirements to prevent such collisions, particularly through implementing first-fault safety. For example, the European standard IEC 60601, which is relevant for electrically operated medical devices, includes specific requirements in this regard.

First-fault safety is typically achieved by implementing a signal path alongside an independently operating safety path, also referred to as the control path. The signal path is often also referred to as the C path and the safety path as the P path. On the signal path side, control signals are generated via a control facility, which cause the unit to move and are transmitted to corresponding actuators. On the safety path side, captured measurement data relating to an actual motion of the unit is sent to a monitoring facility by which, in the event of a related error being detected, generates and outputs a signal that interrupts the motion of the unit. Herein, first-fault safety is achieved by ensuring that the components integrated into the respective path operate independently of one another, so that, if an error occurs in the sphere of one of the paths, a reaction directed toward reducing the risk of collision, such as, for example, stopping the motion, is still triggered. In other words, an error in the region of one of the paths does not propagate to the respective other path. A simple example in this regard relates to an emergency stop switch integrated into the safety path, the actuation of which immediately stops the unit. The signal generated via the emergency stop switch leads directly and independently of the control signals generated via the control facility to an interruption of the respective motion. A further concept for implementing first-fault safety in which data from 3D cameras is used as measurement data is known from US 2006/0 058 919 A1 and from the as yet unpublished European patent application with the official file number EP 23200297.2.

Mention should also be made of the publication US 2023/0 206 501 A1 which describes the use of a 3D camera in a medical installation. Specifically, the concept described therein relates to calibration relating to coordinate systems assigned to a 3D camera and an imaging unit of the medical installation.

### SUMMARY

An object of one or more example embodiments of the present invention is to provide an improved concept for monitoring a motion of a medical installation using three-dimensional image data, in particular with regard to implementing first-fault safety.

According to one or more example embodiments of the present invention, at least this object is achieved with a method of the type mentioned in the introduction comprising the following steps:
(i) generating the motion of the unit via at least one actuator by generating control signals directed toward this motion via a control facility (also referred to as a controller or control device) and transmitting them to the actuator or a component provided to actuate the actuator via a transmission line of a signal path,
(ii) generating motion signals based on an actual motion of the unit via a motion capturing facility (also referred to as a motion capturing device) and transmitting the motion signals to the control facility via the transmission line or a further transmission line of the signal path,
(iii) checking via the control facility whether there is a deviation between an actual motion of the unit, which is determined based on the motion signals, and a motion of the unit, which is to be expected based on the motion signals,
(iv) generating error signals via the control facility if the check carried out in step (iii) has revealed the presence of such a deviation, wherein the error signals are directed toward reducing or eliminating a risk potentially emanating from the motion of the unit,
(v) capturing three-dimensional image data relating to the unit via an image capturing facility (also referred to as an image capturing device), wherein the image data is transmitted to a monitoring facility (also referred to as a monitoring device) via a transmission line of a safety path,
(vi) generating expectation signals via the control facility, wherein the expectation signals relate to the motion of the unit to be generated, and transmitting the expectation signals to the monitoring facility,
(vii) checking via the monitoring facility whether there is a deviation between an actual motion of the unit, which is determined based on the image data, and a motion of the unit, which is to be expected based on the expectation signals,
(viii) generating error signals via the monitoring facility if the check carried out in step (vii) has revealed the presence of such a deviation, wherein the error signals are directed toward reducing or eliminating a risk potentially emanating from the motion of the unit.

In the context of an example embodiment of the present invention, two parallel method strands are performed independently of one another, wherein one of the method strands implements the signal path and the other method strand implements the safety path. While steps (i) to (iv) relate to the signal path, steps (v) to (viii) relate to the safety path. The components provided in this respect, i.e. the control facility and the motion capturing facility with regard to the control path and the monitoring facility and the image capturing facility with regard to the safety path, are present as separate units that operate independently of one another, so that, in the event of the occurrence of an error in one of these units, only the respective path is affected, but not the other path. Steps (i) to (iv) and steps (v) to (viii) are preferably performed parallel to one another, i.e. in particular simultaneously.

In the first step (i), the control signals initiating the motion are generated via the control facility and output to the at least one actuator or to the component provided to actuate the actuator. Transmission can take place via a data bus, for example a CAN bus. The control signals can be generated on the basis of an implementation rule, in particular a fixed implementation rule, such as, for example, a flowchart or flow protocol. It is also conceivable that the control signals are generated based on user signals specified by a user. This specification can be made via a suitable man-machine interface, for example a joystick.

In the second step (ii), the motion signals are generated that relate to an actual motion, i.e. a motion of the unit that is actually physically present and captured by measurement technology. Thus, depending on a current motion, i.e., for example, a current position and/or speed, measurement signals are generated which are motion signals or from which the motion signals are determined. For this purpose, the technical measurement can be directly related to the unit itself. The technical measurement can also relate to the motion of the unit indirectly and, for example, take place by capturing a motion on the part of the actuator or a component connecting the actuator to the unit.

In the third step (iii), the control facility checks whether the measured motion of the unit and the control signals directed toward the motion of the unit are consistent with each other. If this check performed by the signal path results in a match or no deviation, the signal path assumes that the motion is free of errors, so that an intervention in this regard, which would otherwise be performed in step (iv), is not necessary. In this case, the control signals are further generated in such a way that the motion of the unit is continued in accordance with the implementation rule or the user specification. Otherwise, in step (iv), the error signals are generated as an intervention measure directed toward reducing or eliminating a potential risk emanating from the motion of the unit or causing this. Specific details in this regard will be explained later.

In the fifth step (v), which represents a first step of the method strand provided for implementing the safety path, the three-dimensional image data is captured via the image capturing facility. The image capturing facility can be or comprise at least one 3D camera. The image capturing facility is in particular configured to capture data relating to the visible part of the electromagnetic spectrum. Additionally or alternatively, the image capturing facility can be or comprise a facility (or device) for capturing infrared light and/or a lidar facility (also referred to as a lidar device). The three-dimensional image data maps the unit and its surroundings. The image capturing facility or the 3D camera can be arranged on the top of the medical installation, wherein the corresponding field of view is directed downward. Preferably, the image capturing facility comprises a plurality of 3D cameras with different fields of view, wherein the complete region of the medical installation is captured by the fields of view and all conceivable positions of the unit are contained in at least one of the fields of view.

The image data can be present as a two-dimensional array of pixels, wherein each pixel is assigned a depth value relating to the distance between the component mapped therein and the image capturing facility. It is also conceivable that the image data is provided as a three-dimensional array of voxels. The monitoring facility is embodied and configured to process and evaluate the image data. Thus, image evaluation software suitable for this purpose can be implemented on the monitoring facility. In particular, in the context of the evaluation of the image data, the image data is segmented so that regions present in the image data in the unit are assigned to the unit or other components located in the region of the medical installation. In any case, the image data is transmitted to the monitoring facility via a transmission line which is a different transmission line with regard to the transmission of the control signals from the control facility to the actuator or the component provided to actuate the actuator and the motion signals from the motion capturing facility to the control facility.

In the sixth step (vi), which represents a second step of the method strand provided for implementing the safety path, the expectation signals are generated via the control facility and transmitted to the monitoring facility. This transmission preferably takes place via a transmission line separate from the other transmission lines. Preferably, the expectation signals are not the control signals themselves, but, like the control signals, are generated based on the implementation rule or the user specification, so that the expectation signals are not influenced by any error that occurs during the generation of the control signals.

In the seventh step (vii), which represents a third step of the method strand provided for implementing the safety path, the monitoring facility checks whether the motion of the unit ascertained based on the image data and the expectation signals directed toward the expected motion of the unit are consistent with each other. If this check performed by the safety path results in a match in this regard or no deviation, the safety path assumes that the motion is free of errors, so that a corresponding intervention, which would otherwise be performed in step (viii), is not necessary. In this case, the error signals are not generated, so that the motion of the unit can be continued. Otherwise, in step (viii), which represents a fourth step of the method strand provided for implementing the safety path, the monitoring facility generates error signals, which, generally speaking, are directed toward reducing or eliminating a potential risk emanating from the motion of the unit or cause this, as a corresponding intervention measure. Specific details in this regard will also be explained later.

In steps (iii) and (vii), two comparison variables are checked, wherein error-free performance of the motion of the unit in the context of the respective path is only assumed if they match. While, in the third step (iii) in this regard the comparison variables are generated based on the motion signals on the one hand and based on the control signals on the other, in the seventh step (vii), the comparison variables are generated based on the image data on the one hand and based on the expectation signals on the other.

The comparison variables ascertained can be motion information relating to or describing a position and/or a speed and/or an acceleration of the unit. In this case, a reference system that is fixed with respect to the medical installation, the image capturing facility or the unit can be used. In this case, it is also possible for a plurality of such reference systems to be used, wherein coordinate transformations can be performed to enable comparability.

With regard to the third step (iii), a first item of motion information and a second item of motion information can be ascertained as the comparison variables. The first item of motion information is ascertained based on the control signals and relates to the motion of the unit as would be expected in error-free operation based on the control signals. The second item of motion information is ascertained based on the motion signals and relates to the actual or real motion of the unit.

With regard to the seventh step (vii), a third item of motion information and a fourth item of motion information can be ascertained as the comparison variables. The third item of motion information is ascertained based on the image data and relates to the actual or real motion of the unit. The third item of motion information can be ascertained by evaluating the image data with respect to at least one reference point of the unit, in particular with the additional use of known dimensions and geometries of the unit. The reference points provided can be markings applied to the unit or prominent locations on the unit. The fourth item of motion information is ascertained based on the expectation signals and relates to the motion of the unit as would be expected with error-free operation based on the control signals.

One of the key advantages of the present invention relates to the conceivably simple implementation of the safety path. Thus, in this regard, a component of the medical installation that is frequently anyway provided, namely the image capturing facility, is used as the capturing mechanism for the measurement data. Thus, 3D cameras are already frequently used at present, for example in the course of imaging or treatment, for example for the purpose of measuring or determining the position of the patient. In the prior art, an encoding facility (also referred to as an encoding device) for implementing the signal is often provided, wherein a further encoding facility for implementing the safety path is provided. With the present invention, there is no need for the further encoding facility, so that there is also no need for corresponding cabling.

It is conceivable that at least one motor controller implementing a frequency converter is provided by which the electromechanical actuator connected thereto can be supplied with an operating voltage required to generate the motion of the unit, wherein the control signals generated in step (i) for controlling the actuator are transmitted to the motor controller. In principle, the actuator can in particular be a three-phase motor. The motor controller connects a voltage source to the actuator, wherein an amplitude and/or a frequency of the voltage applied to the actuator is controlled based on the control signals. For this purpose, the motor controller has an interface that is connected to the transmission line via which the control signals are transmitted. Alternatively, the actuator can also be embodied as a DC motor or a stepper motor.

In the method according to an example embodiment of the present invention, it can be provided that the motion capturing facility is an encoding facility by which the actual motion of the unit and/or the actuator is converted into the motion signals in step (ii). The encoding facility can also be referred to as an encoder. The motion signals or measurement signals from which the motion signals are ascertained are generated via the encoding facility based on the current position of the unit or a component connected thereto. Thus, in this regard, the current position of a push rod with respect to a linear motion or a joint with respect to its swivel position can be ascertained.

Preferably, it is provided according to an example embodiment of the present invention that the motion of the unit is generated via a plurality of actuators by generating control signals which are transmitted to the actuators or the components provided to actuate the actuators, so that the motion is an overall motion which is composed of individual motions which are in each case generated via the actuators. In the context of this embodiment, a so-called multi-axis motion can be implemented, wherein the individual motions can in each case be linear motions or swivel motions that add up to the overall motion and, for example, enable the unit to move along a curved trajectory. In the context of this embodiment, it is checked in step (vii) whether there is a deviation between the actual overall motion of the unit, which is determined based on the image data, and the overall motion of the unit, which is to be expected based on the expectation signals.

Particularly preferably, the error signals generated in step (iv) or (viii) cause the motion of the unit to stop. Thus, stopping this motion, i.e. reducing the speed of the unit to zero, is an effective measure for eliminating any risk present due to the motion. Specifically, the error signals generated in step (iv) by the control facility can be control signals that cause the motion of the unit to stop. Therefore, the control facility actively intervenes in the control process. It is also conceivable that the error signals generated in step (viii) are generated by the monitoring facility and output to an interrupting facility (also referred to as an interrupting device) and/or a braking device and cause an interruption of the application of the operating voltage to the actuator via the interrupting facility and/or a locking of the unit via the braking device. The interrupting facility is preferably a switch by which the power supply to the actuator can be interrupted. In particular to prevent any further motion of the unit due to inertia and/or gravity, the braking device immobilizes the unit. For this purpose, the braking device can have brake discs and/or brake shoes that interact accordingly with an element moving during the motion of the unit via frictional forces.

It is conceivable that the error signals generated in step (iv) or step (viii) are transmitted to an output facility (also referred to as an output device) and cause the output of an error message via the output facility, by which a user is informed of the presence of a potential fault situation. The output facility can be configured to output a visual and/or acoustic error message. Thus, the output facility can comprise a display or touchscreen and/or loudspeaker. The error message can be output as text. Thus, the user can, for example, be informed accordingly via the text “Caution, potential fault or risk of collision during motion”, so that the user can also take countermeasures if necessary, such as, for example, actuating an emergency stop switch integrated into a further safety path.

If the respective error signals cause both the stoppage of the motion of the unit and the output of the error message, then, after the generation of the error signals causing the stoppage of the motion of the unit and the output of the error message, the fulfillment of a release condition can also be checked, wherein the release condition is fulfilled if a release signal generated via an input facility (also referred to as an input device) is present, wherein the generation of the release signal takes place via a user action performed on the input facility and directed toward the release of the motion, wherein the motion of the unit is only continued on the fulfillment of the release condition, in particular in the context of reduced speed. This embodiment relates to the case in which the user, i.e. specialist personnel trained with respect to the operation of the medical installation, nevertheless wishes to continue the respective motion in the event of an error detected by the signal path and/or the safety path. This can be the case if the user recognizes that, despite the presence of an error, there is no risk of collision from the moving unit.

It is preferably provided that the image data is transmitted to the control facility, wherein the control signals are generated, in particular additionally, based on the image data. In this case, the image data is used synergistically not only to implement the safety path, but also for a further purpose, namely to generate the control signals.

Thus, the image data can be evaluated by the control facility in such a way that in this regard image-based motion information relating to the unit is captured, wherein the image-based motion information is compared with motion information that was ascertained based on the motion signals and which has already been referred to above as a second item of motion information. If these items of motion information are compared, and the ascertained deviation is less than a predefined limit value, it can be assumed that no error is currently present and that the control signals relating to the motion of the unit can continue to be generated and output. This implements a further safety level on the part of the signal path. In principle, in this regard, motion control of the movable unit can be carried out in accordance with European patent application EP 23200297.2 already mentioned in the introduction, to which reference is made accordingly.

Specifically, it is also conceivable that, via the control facility and based on the image data, at least one item of unit information relating to a current position and/or motion of the unit and one item of object information relating to a current position and/or motion of an object located in the region of the medical installation is ascertained, wherein the control signals are generated based on the at least one item of unit information and/or the at least one item of object information. The object can be any object, including the patient, and/or any unit of the medical installation located in the region of the medical installation. The object can be stationary with respect to the medical installation. However, the object can also be a further movable unit of the medical facility (also referred to as a medical device). The corresponding data evaluation is in particular carried out based on the segmentation of the image data already mentioned above.

It is conceivable that the control facility is used to check the fulfillment of a collision condition, which is fulfilled if the at least one item of unit information and the at least one item of object information indicate that there is currently a risk of collision between the unit and the object, wherein, if the collision condition is fulfilled, the control signals are generated via the control facility in such a way that the risk is reduced or eliminated. Thus, probable trajectories of the unit and/or the object can be ascertained based on the unit information and/or the object information, wherein the collision condition is fulfilled if these trajectories have a point of intersection or, generally speaking, a common region. In particular if the dimensions and geometries of the unit and/or of the object are known, the trajectories can be determined as the volume regions of the region of the medical installation that are swept over by the unit or the object. The collision condition can be fulfilled if these volume regions are not disjoint. In addition, for the fulfillment of the collision condition, it is possible to check a temporal condition, which is fulfilled if the probable trajectory/trajectories indicate that the unit and the object will probably be at the same position at the same time.

The present invention further relates to a medical installation, comprising at least one movable unit and a control facility configured to generate control signals directed toward the motion of the unit and causing this motion and to transmit them to an actuator via a transmission line of a signal path and a motion capturing facility by which, based on an actual motion of the unit, motion signals can be generated and transmitted to the control facility via the transmission line or a further transmission line of the signal path, wherein the control facility is furthermore configured to check whether there is a deviation between an actual motion of the unit, which can be determined based on the motion signals, and a motion of the unit, which is to be expected based on the motion signals, and, in the event of the presence of such a deviation, to generate error signals, wherein the error signals are directed toward reducing or eliminating a risk potentially emanating from the motion of the movable unit, wherein the medical installation further comprises an image capturing facility by which three-dimensional image data relating to the unit can be captured and transmitted to a monitoring facility via a transmission line of a safety path, wherein the control facility is further configured to generate expectation signals relating to the motion of the unit to be generated and to output them to the monitoring facility, wherein the monitoring facility is configured to check whether there is a deviation between an actual motion of the unit, which can be determined based on the image data, and a motion of the unit, which is to be expected based on the expectation signals, and, in the event of the presence of such a deviation, to generate error signals, wherein the error signals are directed toward reducing or eliminating a risk potentially emanating from the motion of the unit. All features, advantages and aspects explained in connection with the method according to an example embodiment of the present invention, are equally transferrable to the medical installation according to an example embodiment of the present invention and vice versa.

The medical installation according to an example embodiment of the present invention can be a medical imaging facility. The medical imaging facility can be a magnetic resonance imaging facility (also referred to as a magnetic resonance imaging device) or a computed tomography facility (also referred to as a computed tomography device) or an angiography device or an ultrasound device or a positron emission tomography installation. It is also conceivable that the medical installation according to an example embodiment of the present invention is a treatment facility (also referred to as a treatment device), in particular a radiotherapy installation.

The unit or one of the units can be a patient support table for accommodating a patient for the performance of imaging or treatment or an imaging unit, in particular a C-arm or an apparatus for generating ionizing radiation. In particular the unit is supported by a repositioning apparatus by which the motion of the unit can be generated and which in particular comprises the actuator. The repositioning apparatus can be an articulated or support arm that can be moved via the at least one actuator.

Moreover, the present invention relates to a control device for a medical installation in accordance with the preceding descriptive passage, wherein the control device is embodied as a control facility or a monitoring facility configured to perform the method according to the above description. A computer program can be implemented on the control device by which at least some of the steps of the method according to an example embodiment of the present invention can be performed, in particular automatically. Therefore, the execution of the computer program causes the execution of at least some of the above-described steps (i) to (vii). All advantages, features and aspects explained in connection with the method according to an example embodiment of the present invention and the medical installation according to an example embodiment of the present invention are equally transferrable to the control device according to an example embodiment of the present invention and vice versa.

Example embodiments of the present invention furthermore relate to a computer program, which can be loaded into a memory unit of a control device in accordance with the preceding descriptive passage, comprising program sections for executing steps of the method according to the above description when the computer program is executed by the control device. All advantages, features and aspects explained in connection with the method according to an example embodiment of the present invention, the medical installation according to an example embodiment of the present invention and the control device according to an example embodiment of the present invention are equally transferrable to the computer program according to an example embodiment of the present invention and vice versa.

Finally, embodiments of the present invention relates to a computer-readable medium on which program sections readable and executable by a control device according to the above description are stored in order to execute steps of the method according to the above description when the program sections are executed by the control device. All advantages, features and aspects explained in connection with the method according to an example embodiment of the present invention, the medical installation according to an example embodiment of the present invention, the control device according to an example embodiment of the present invention and the computer program according to an example embodiment of the present invention are equally transferrable to the computer-readable medium and vice versa.

### BRIEF DESCRIPTION OF THE DRAWINGS

Further advantages and details of the present invention emerge from the exemplary embodiments described below and with reference to the figures. The figures show schematically:

FIG. 1: a schematic sketch of a medical installation according to an exemplary embodiment, comprising a control device embodied as a control facility according to an exemplary embodiment and a control device embodied as a monitoring facility according to an exemplary embodiment,

FIG. 2: a schematic representation of the medical installation in FIG. 1,

FIG. 3: a flowchart illustrating a method according to an exemplary embodiment which is performed based on the medical installation shown in FIG. 1, and

FIG. 4: a detailed view of the flowchart in FIG. 3 relating to the first step of this method.

### DETAILED DESCRIPTION

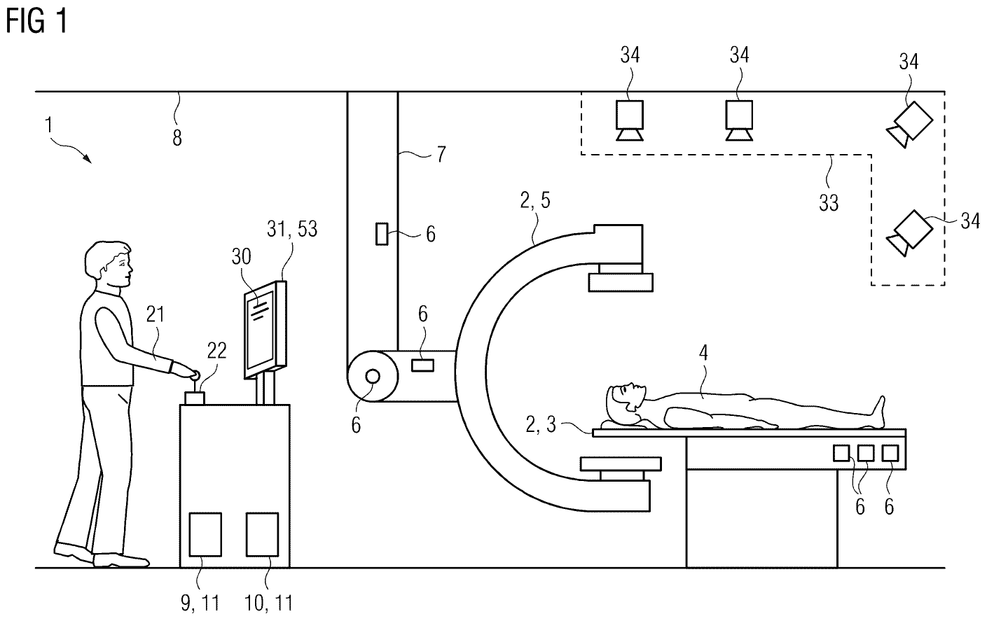

FIG. 1 shows an exemplary embodiment of a medical installation 1, in the present case a medical imaging facility. Alternatively, the medical installation 1 can be a treatment facility. The only essential feature for the present invention is that the medical installation 1 comprises at least one movable unit 2 which can be repositioned or moved during imaging or treatment.

In the medical installation 1 according to the present exemplary embodiment, two such units 2 are provided by way of example, namely a patient support table 3 for accommodating a patient 4 during the performance of imaging and an imaging unit 5 embodied as a C-arm. The patient support table 3 is coupled to three actuators 6 by which a motion of the patient support table 3 can be performed in each case, namely with respect to the up-down, left-right and forward-backward directions. The imaging unit 5 is supported by a repositioning apparatus which is a support arm 7 mounted on the ceiling 8. The support arm 7 also comprises actuators 6 by which translational motions and swivel motions of the imaging unit 5 can be performed.

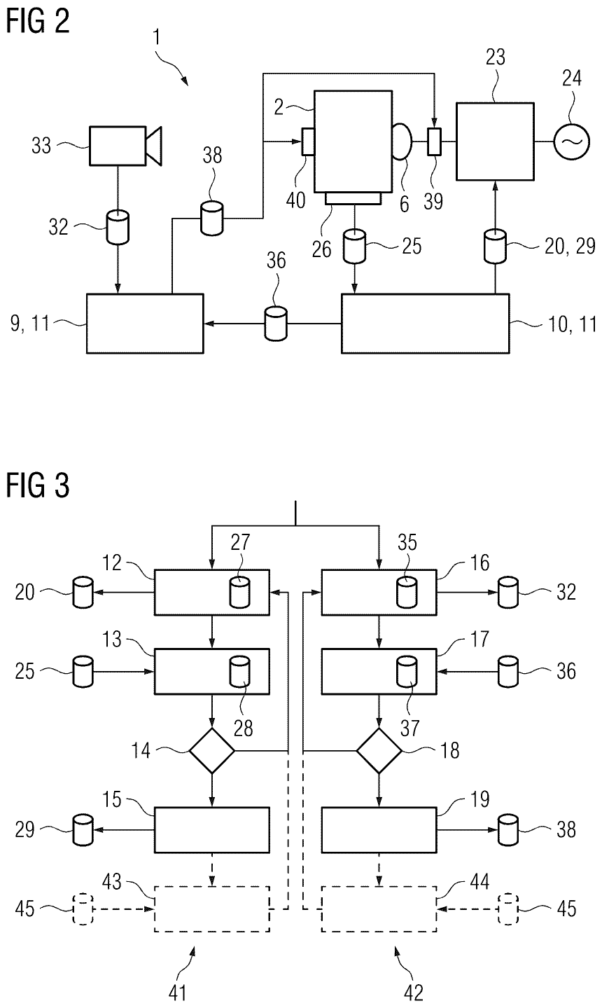

FIG. 2 shows a highly schematic representation of the medical installation 1, wherein transmitted signals are symbolically indicated by cylinders. FIG. 3 shows a flowchart illustrating the method according to an exemplary embodiment; this will be explained below with reference to the medical installation 1 shown in FIGS. 1 and 2. To perform the method according to an example embodiment of the present invention, the medical installation 1 comprises a control facility 9 and a monitoring facility 10, which in each case implement a control device 11 according to an exemplary embodiment in each case. According to an exemplary embodiment, a computer program is in each case provided for each of the control devices 11 and can be loaded into a memory unit of the respective control device 11. Each of the computer programs comprises program sections for executing steps of the method when the computer program is executed by the respective control device 11. The control devices 11 further in each case comprise a inventive computer-readable medium according to an exemplary embodiment on which in each case program sections readable and executable by the respective control device 11 are stored in order to execute steps of the method according to the above description when the program sections are executed by the respective control device 11. The method comprises steps 12 to 19, wherein steps 12 to 15 and 16 to 19, as also correspondingly indicated in FIG. 3, are in each case performed in parallel and simultaneously with one another. Herein, steps 12 to 15 implement a signal path 41 and steps 16 to 19 implement a safety path 42.

In the first step 12 of the method strand provided for implementing the signal path 41, the control facility 9 is used to generate control signals 20 by which the actuators 6 are in each case actuated to generate a desired motion of the respective unit 2. The control signals 20 are transmitted to the respective actuator 6 via a data bus, namely a CAN bus. The control signals 20 are generated either on the basis of a fixed predefined implementation rule, such as, for example, a corresponding flowchart, which is held or stored by the control facility 9. Alternatively, the control signals 20 are generated based on user signals generated by a user 21 via a man-machine interface 22, in the present case a joystick.

The control signals 20 are used to ascertain a first item of motion information 27 relating to or describing the motion of the respective unit 2 as would be expected with error-free operation based on the control signals 20. Specifically, the first item of motion information 27 relates to the corresponding current position and the speed of the respective unit 2. The first item of motion information 27 comprises the coordinates of the respective unit 2 and its speed in terms of magnitude and direction.

With particular reference to FIG. 2, it can be seen that the actuators 6 are controlled indirectly via the control signals 20. For the sake of clarity, FIG. 2 only shows one of the actuators 6, wherein the explanations apply equally to the other actuators 6. Thus, a motor controller 23 implementing a frequency converter is provided for each of the actuators 6 by which the electromechanical actuator 6 connected thereto can be supplied with an operating voltage required to generate the motion of the unit 2 coupled to the respective actuator 6. The control signals 20 are therefore transmitted to the motor controller 23. The actuators 6 are in each case embodied as a three-phase motor. The motor controller 23 connects a voltage source 24 to the respective actuator 6, wherein an amplitude and a frequency of the voltage applied to the respective actuator 6 is controlled based on the control signals 20. It is also conceivable that the actuator 6 is a DC motor or a stepper motor.

In the second step 13 of the method strand provided for implementing the signal path 41, motion signals 25 are generated via a motion capturing facility 26. Like the transmission of the control signals 20 to the actuator 6 or to the motor controller 23, the motion signals 25 are transmitted to the control facility 9 via a corresponding transmission line of the signal path 41. The motion capturing facility 26 is an encoding facility or an encoder by which the actual motion of the unit 2 or the actuator 6 connected to the unit 2 can be captured by measuring technology. Based on the current position of the unit 2 or a component of the actuator 6 connected thereto, the motion capturing facility 26 generates the measurement signals from which the motion signals 25 are ascertained or which are the motion signals 25. Depending on which of the actuators 6 is specifically involved in the present case, in this regard, the current position is ascertained with respect to the translational-linear motion of a push rod or the swivel motion of a joint.

The control facility 9 ascertains a second item of motion information 28 based on the motion signals 25. The second item of motion information 28 relates to the actual or real motion of the unit 2. The second item of motion information 28 comprises the coordinates of the respective unit 2 and its speed in terms of magnitude and direction.

In the third step 14 of the method strand provided for implementing the signal path 41, the control facility 9 checks whether there is a deviation between an actual motion of the unit 2, which is determined based on the motion signals 25, and a motion of the unit 2, which is to be expected based on the control signals 20. Herein, specifically the first item of motion information 27 is compared with the second item of motion information 28 with regard to the presence of a match. In this regard, it is conceivable that an error situation is assumed if the deviations of the coordinates and/or the speed are greater than a respective, in particular fixed, predefined limit value. If this is not the case, the method is continued again in the first step 12 of the method strand provided for implementing the signal path 41 with the generation of the control signals 20 and the subsequent capture of the motion signals 25.

If the check carried out in step 14 has revealed that there is a deviation in this regard, error signals 29 are generated via the control facility 9 in the fourth step 15 of the method strand provided for implementing the signal path 41. Although the presence of a deviation could in principle be attributable to an error on the part of the motion capturing facility 26, it is also possible that the current motion does not correspond to that which should occur during the current operation of the medical installation 1 and in accordance with the implementation rule or the user signals. In this case, the moving unit 2 could potentially pose a risk. The error signals 29 are directed toward reducing or eliminating this risk.

On the one hand, the error signals 29 cause the motion of the unit 2 to stop. Thus, the error signals 29 represent control signals 20 that result in the motion of the unit 2 being stopped. In addition, the error signals 29 cause an error message 30 to be output via an output facility 31 of the medical installation 1 embodied as a touchscreen. Here, the user 21 is shown a warning text or an acoustic warning is output which indicates the possible presence of a current malfunction with respect to the motion.

In parallel to steps 12 to 15 explained above, in the first step 16 of the method strand provided for implementing the safety path 42, three-dimensional image data 32 is captured and transmitted via a transmission line of the safety path 42 to the monitoring facility 10. The image data 32 relates to the region of the medical installation 1 and therefore sometimes shows the unit 2. To capture the image data 32, an image capturing facility 33 arranged on the ceiling 8 is provided; this comprises a plurality of 3D cameras 34. The fields of view of the 3D cameras 34 are different from one another and directed downward so that the entire region of the medical installation is captured. At least all conceivable positions of the units 2 are captured by the field of view of at least one of the 3D cameras 34.

The image data 32 captured by one of the 3D cameras 34 is available as a two-dimensional array of pixels, wherein each pixel is assigned a depth value which assigns a distance to the respective 3D camera 34. The image data 32 is evaluated via the monitoring facility 10 on which appropriate image evaluation software is implemented. Here, the image data 32 is sometimes segmented so that the regions present in the image data 32 are assigned to the units 2 and further components and objects. This image data 32 is used to ascertain a third item of motion information 35 via the monitoring facility 10. Here, the image data 32 is evaluated with regard to the detection of reference points on the unit 2, for example markings applied to the unit 2 and/or prominent locations on the unit 2, for example corners or edges. Known dimensions and geometries of the unit 2 are also used here. The third item of motion information 35 comprises the coordinates of the respective unit 2 and its speed in terms of magnitude and direction.

In the second step 17 of the method strand provided for implementing the safety path 42, the control facility 9 generates expectation signals 36 and outputs them to the monitoring facility 10. Like the control signals 20, the expectation signals 36 are generated based on the motion of the unit 2 to be generated. From the expectation signals 36, the monitoring facility 10 ascertains a fourth item of motion information 37, which indicates the coordinates of the respective unit 2 to be expected and its expected speed when the control signals 20 are executed without errors.

In the third step 18 of the method strand provided for implementing the safety path 42, the monitoring facility 10 checks whether there is a deviation between the actual motion of the respective unit 2, which was determined based on the image data 32, and the motion of the respective unit 2, which is to be expected based on the expectation signals 36. Here, specifically, the third item of motion information 35 is compared with the fourth item of motion information 37 with regard to whether they match. In this regard, it is conceivable that an error situation is assumed if the deviations of the coordinates and/or the speed are greater than a respective, in particular fixed, predefined limit value. If this not the case, the method is continued again in the first step 16 of the method strand provided for implementing the safety path 42 with the capture of the image data 32 and the subsequent capture of the expectation signals 36.

If the check in the third step 18 of the method strand provided for implementing the safety path 42 has indicated that there is a deviation in this regard, error signals 38 are generated via the monitoring facility 10 in the fourth step 19 of the method strand provided for implementing the safety path 42. Although the presence of a deviation could in principle be attributable to an error on the part of the image capturing facility 33, for example since data from a frozen image is supplied, it is possible that the motion currently present does not correspond to that which should occur in the course of the current operation of the medical installation 1. Therefore, in this case once again, there is potentially a risk emanating from the moving unit 2. Like the error signals 29 generated via the control facility 9, the error signals 38 generated via the monitoring facility 10 are directed toward reducing or eliminating this risk.

Thus, the error signals 38 cause the motion of the unit 2 to stop. For this purpose, the error signals 38 are output to an interrupting facility 39 and a braking device 40. On the part of the interrupting facility 39, the error signals 38 cause an interruption to the supply of the operating voltage to the respective actuator 6. The interrupting facility 39 is embodied as a switch that can be actuated by the error signals 38 which interrupts the power supply to the respective actuator 6 when the corresponding error signals 38 are present. The braking device 40 has braking apparatuses, such as, for example, brake discs and/or brake shoes that are triggered when error signals 38 are present. Here, braking forces are generated on any component of the respective unit 2 or the respective actuator 6 that is still moving, so that any motions of the unit 2 that are still present due to inertia or gravity are braked and stopped. In addition, the error signals 38 cause the output of the error message 30 that has already been explained in connection with the fourth step 15 of the method strand provided for implementing the signal path 41 via the output facility 31.

The above-explained steps 12 to 19 implement first-fault safety in the medical installation 1 by implementing a signal path 41 via steps 12 to 15 and a safety path 42 via steps 16 to 19. The components used in the course of the implementation of the paths 41, 42, in particular the monitoring facility 10 and the control facility 9 and the transmission lines provided for the signal transmission of the respective paths 41, 42 are embodied as separate components that operate independently of one another so that an error present in one of the paths 41, 42 does not propagate to the respective other path 41, 42. With regard to the error signals 29, 38, the specific implementation of the two paths 41, 42 creates redundancy.

As already mentioned, the medical installation 1 comprises a plurality of movable units 2, namely the patient table 3 and the imaging unit 5. The above-explained steps 12 to 19 are performed separately for each of the units 2, so that the motion monitoring is performed with respect to all movable units 2. It is also provided that the motion of the respective unit 2 is accomplished via a plurality of actuators 6, so that the motion of the respective unit 2 is an overall motion composed of individual motions generated in each case via one of the actuators 6. This enables multi-axis motions of the units 2. Here, the individual motions are linear motions or swivel motions that add up to the overall motion and, for example, enable a curved trajectory for the respective unit 2. In the context of this embodiment, in the third step 18 of the method strand provided for implementing the safety path 42, it is checked whether there is a deviation between the actual overall motion of the respective unit 2, which is determined based on the image data 32, and the overall motion of the respective unit 2, which is to be expected based on the expectation signals 36.

Further, it should be noted that steps 12 to 15 and steps 16 to 19 are performed simultaneously and parallel to one another, wherein, if the check in the third step 14 of the method strand provided for implementing the signal path 41 or in step 18 indicates that there is a deviation, each of these two method strands is started again in the first step 12, 16 for the respective method strand. Steps 12 to 14 and 16 to 18 are therefore run through cyclically, wherein the signals or data 20, 45, 32, 36 are continuously recaptured and therefore updated. The time required to performed these cycles can be a maximum of 1 second, for example a tenth of second.

The following explains further optional aspects of the method with regard to the fourth step 15 of the method strand provided for implementing the signal path 41 and the fourth step 19 of the method strand provided for implementing the safety path 42. Thus, in steps 15, 19, in each case error signals 29, 38 are generated and output which, in addition to directly stopping the motion of the respective unit 2, cause the additional output of the error message 30 to the user 21. In this situation, it is conceivable that a technician is consulted to check which of the components of the medical installation 1 is affected by the error and to rectify it if necessary. In this case, the respective method strand can be restarted from the beginning. However, it is also conceivable that the user 21 already recognizes that actually there is currently no error situation or no risk with respect to the motion of the unit 2, so that, for example to avoid additional stress for the patient 4, it is advisable to continue the operation of the medical installation 1 or the motion of the respective unit 2. Steps 43, 44, which are explained below and optionally follow steps 15, 19, are directed toward this. In the flowchart shown in FIG. 3, these optional steps 43, 44 are indicated by dashed lines.

In the respective step 43, 44, a release condition is checked, wherein, for this purpose, the control facility 9 is configured and provided with regard to step 43 and the monitoring facility 10 is configured and provided with regard to step 44. Thus, in addition to the error message 30, a button is displayed to the user 21 via the output facility 31 on which the text “Continue process anyway” is displayed. The user 21 can select the button by clicking or tapping it. The corresponding selection of this button causes a release signal 45 to be generated, the presence of which fulfills the release condition. The output facility 31 therefore also functions as an input facility 53 of the medical installation 1. The release signal 45, which is transmitted to the control facility 9 or the monitoring facility 10, causes the method to be continued in the context of the first step 12 of the method strand provided for implementing the signal path 41 or the first step 16 of the method strand provided for implementing the safety path 42. Consequently, the fulfillment of the release condition causes the motion of the respective unit 2 to continue, in the present case with reduced speed only, by way of example. It is also conceivable that steps 12 to 15 or 16 to 19 of the respective faulty path 41, 42 are no longer carried out or are suppressed when the release condition is fulfilled.

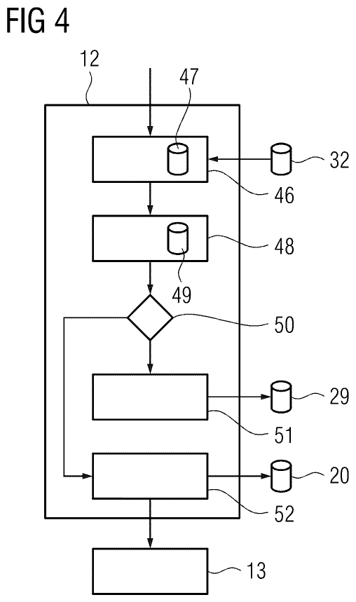

The following explains a conceivable aspect with reference to FIG. 4 with regard to the first step 12 of the method strand provided for implementing the signal path 41. Thus, FIG. 4 shows further details with regard to step 12 as a flowchart. Thus, in a step 46, the image data 32 is transmitted to the control facility 9 and, as explained below, is used to generate the control signals 20. Thus, the control facility 9 ascertains, based on the image data 32, unit information 47 relating to the current motion, i.e. the current position and the current speed, of the respective unit 2, wherein in this regard the same procedure is provided as for ascertaining the third item of motion information 35 via the monitoring facility 10.

In the following step 48, object information 49 is ascertained based on the image data 32; this relates to a current position and/or motion of an object located in the region of the medical installation 1, which is also mapped in the image data 32. The object can be stationary or moving in the region of the medical installation 1. Here, once again, in this regard the same or an analogous evaluation of the image data 32 is carried out with regard to the object as that for ascertaining the motion information 35 or the unit information 47.

In the next step 50, the fulfillment of a collision condition is checked; this is fulfilled if the unit information 47 and the object information 49 indicate that there is currently the risk of collision between respective unit 2 and the respective object. For this purpose, unit information 47 and the object information 49 are used to ascertain probable trajectories of the respective unit 2 and, if applicable, the object, wherein the collision condition is fulfilled if these trajectories have a point of intersection or, generally speaking, a common region. Here, use is also made of known dimensions and geometries of the unit 2 and, if applicable, the object. Accordingly, the trajectory is present as the volume region of the region of the medical installation 1 that is swept over by the unit 2 or the object. If the object is stationary, there is no trajectory in this regard, but instead the corresponding volume region of the region of the medical installation 1 in which the object is located or which is filled by the object. The collision condition is fulfilled when these volume regions are not disjoint. Additionally, a temporal condition is checked to check the fulfillment of the collision condition; the temporal condition is fulfilled if the probable trajectories indicate that the respective unit 2 and the object will be at the same position or the point of intersection at the same time.

If the collision condition is fulfilled, the control signals 20 are generated in such a way that the risk with respect to this collision is reduced or eliminated. Accordingly, in this case, the procedure explained above with reference to the above-described fourth step 15 of the method strand provided for implementing the signal path 41 can be followed, so that corresponding error signals 29 are generated and output in the next step 51. Specifically, in this case, a procedure as described in the European patent application with the file reference EP 23200297.2 can also be provided. If the collision condition is not fulfilled, the control signals 20 are generated in the context of the next step 52 based on the implementation rule and/or the user signals and output to the actuator 6 or the motor controller 23.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java (0), Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for monitoring motion of at least one movable unit of a medical installation, the method comprising:
generating motion of the at least one movable unit via at least one actuator in that control signals directed toward the motion are generated via a control device and transmitted to the at least one actuator or a component provided to actuate the at least one actuator via a transmission line of a signal path;
generating motion signals based on an actual motion of the at least one movable unit, via a motion capturing device, and transmitting the motion signals to the control device via the transmission line or a further transmission line of the signal path;
first checking, via the control device, whether there is a first deviation between the actual motion of the at least one movable unit, which is determined based on the motion signals, and a first expected motion of the at least one movable unit, the first expected motion being based on the control signals;
generating first error signals via the control device in response to the first checking indicating a presence of the first deviation, wherein the first error signals are directed toward reducing or eliminating a risk potentially emanating from the motion of the at least one movable unit;
capturing three-dimensional image data relating to the at least one movable unit, via an image capturing device, wherein the three-dimensional image data is transmitted to a monitoring device via a transmission line of a safety path;
generating expectation signals, via the control device, wherein the expectation signals relate to the motion of the at least one movable unit to be generated, and transmitting the expectation signals to the monitoring device;
second checking, via the monitoring device, whether there is a second deviation between an actual motion of the at least one movable unit, which is determined based on the three-dimensional image data, and a second expected motion of the at least one movable unit, which is based on the expectation signals; and
generating second error signals, via the monitoring device, in response to the second checking indicating the presence of the second deviation, wherein the second error signals are directed toward reducing or eliminating the risk potentially emanating from the motion of the at least one movable unit.

2. The method as claimed in claim 1, wherein
at least one motor controller implementing a frequency converter is provided by which the at least one actuator connected thereto is supplied with an operating voltage required to generate the motion of the at least one movable unit, and
the control signals for controlling the at least one actuator are transmitted to the at least one motor controller.

3. The method as claimed in claim 1, wherein the motion capturing device is an encoding device by which at least one of the actual motion of the at least one movable unit or n actual motion of the at least one actuator is converted into the motion signals to generate the motion signals.

4. The method as claimed in claim 1, wherein
the motion of the at least one movable unit is generated via a plurality of actuators by generating the control signals which are transmitted to the plurality of actuators or components provided to actuate the plurality of actuators, so that the motion is an overall motion which is composed of individual motions that are, in each case, generated via the plurality of actuators, and
the second deviation is a deviation between an actual overall motion of the at least one movable unit, which is based on the three-dimensional image data, and an expected overall motion of the at least one movable unit, which is based on the expectation signals.

5. The method as claimed in claim 1, wherein the first error signals or the second error signals cause the motion of the at least one movable unit to stop, and wherein at least one of
the first error signals are control signals generated by the control device that cause the motion of the at least one movable unit to stop, or
the second error signals are generated by the monitoring device and output to at least one of an interrupting device or a braking device to cause at least one of (i) an interruption, effected via the interrupting device, to supply of an operating voltage required to generate the motion of the at least one movable unit or an operating voltage to the at least one actuator, or (ii) a locking of the at least one movable unit via the braking device.

6. The method as claimed in claim 1, wherein the first error signals or the second error signals are transmitted to an output device and cause output of an error message, via the output device, to inform a user of a potential fault situation.

7. The method as claimed in claim 6, wherein, after the first error signals or the second error signals causing the motion of the at least one movable unit to stop and the error message to be stopped, the method includes checking fulfillment of a release condition, wherein
the release condition is fulfilled when a release signal, generated via an input device, is present,
the release signal is generated via a user action performed at the input device and directed toward release of the motion, and
the motion of the at least one movable unit only continues when the release condition is fulfilled.

8. The method as claimed in claim 1, wherein the three-dimensional image data is transmitted to the control device, and wherein the control signals are generated based on the three-dimensional image data.

9. The method as claimed in claim 8, wherein
via the control device and based on the three-dimensional image data, at least one item of unit information relating to at least one of a current position or motion of the at least one movable unit and at least one item of object information relating to at least one of a current position or movement of an object located in a region of the medical installation is ascertained,
the control signals are generated based on at least one of the at least one item of unit information or the at least one item of object information,
via the control device, fulfillment of a collision condition is checked, the collision condition being fulfilled when the at least one item of unit information and the at least one item of object information reveals that there is currently a risk of a collision between the at least one movable unit and the object, and
when the collision condition is fulfilled, the control signals are generated via the control device such that the risk is reduced or eliminated.

10. A medical installation, comprising:
at least one movable unit; and
a control device configured to
generate control signals directed toward motion of the at least one movable unit and causing the motion,
transmit the control signals, via a transmission line of a signal path, to at least one actuator or a component provided to actuate the at least one actuator, and to a motion capturing device by which, based on an actual motion of the at least one movable unit, motion signals are generated and transmitted to the control device via the transmission line or a further transmission line of the signal path,
check for a first deviation between the actual motion of the at least one movable unit, which is determined based on the motion signals, and a first expected motion of the at least one movable unit, which is based on the control signals, and
in response to the first deviation, generate first error signals, wherein the first error signals are directed toward reducing or eliminating a risk potentially emanating from the motion of the at least one movable unit;
an image capturing device configured to capture three-dimensional image data relating to the at least one movable unit, and transmit the three-dimensional image data to a monitoring device of the medical installation via a transmission line of a safety path, wherein
the control device is further configured to generate expectation signals relating to the motion of the at least one movable unit to be generated, and output the expectation signals to the monitoring device, and
the monitoring device is configured to
check for a second deviation between an actual motion of the at least one movable unit, which is determined based on the three-dimensional image data, and a second expected motion of the at least one movable unit, which is based on the expectation signals, and
in a presence of the second deviation, generate second error signals, wherein the second error signals are directed toward reducing or eliminating the risk potentially emanating from the motion of the at least one movable unit.

11. The medical installation as claimed in claim 10, wherein the medical installation is a medical imaging device.

12. The medical installation as claimed in claim 11, wherein the at least one movable unit includes at least one of a patient support table or an imaging unit, the patient support table being configured to accommodate a patient for imaging or treatment.

13. A control device for a medical installation, wherein the control device is configured to perform the method as claimed in claim 1.

14. A non-transitory computer-readable medium, storing computer-readable instructions that, when executed by a control device, cause the control device to perform the method as claimed in claim 1.

15. The method of claim 7, wherein the release condition is fulfilled in a context of reduced speed.

16. The medical installation as claimed in claim 11, wherein the medical imaging device is a magnetic resonance imaging device, a computed tomography device, an angiography device, an ultrasound device, a positron emission tomography installation, or a treatment device.

17. The medical installation as claimed in claim 16, wherein the treatment device is a radiotherapy installation.

18. The medical installation as claimed in claim 12, wherein the imaging unit is a C-arm or an apparatus configured to generate ionizing radiation.

19. The method as claimed in claim 2, wherein the motion capturing device is an encoding device by which at least one of the actual motion of the at least one movable unit or an actual motion of the at least one actuator is converted into the motion signals to generate the motion signals.

20. The method as claimed in claim 19, wherein
the motion of the at least one movable unit is generated via a plurality of actuators by generating the control signals which are transmitted to the plurality of actuators or components provided to actuate the plurality of actuators, so that the motion is an overall motion which is composed of individual motions that are, in each case, generated via the plurality of actuators, and
the second deviation is a deviation between an actual overall motion of the at least one movable unit, which is based on the three-dimensional image data, and an expected overall motion of the at least one movable unit, which is based on the expectation signals.
